(19) United States
(12) Patent Application Publication
(10) Pub. No.: US 2024/0417759 A1
(43) Pub. Date: Dec. 19, 2024

(54) PROCESSES FOR FIXATION OF CARBON DIOXIDE

(71) Applicant: Jupeng Bio (HK) Limited, Hong Kong (CN)

(72) Inventors: Ryan Senaratne, Fayetteville, AR (US); Bruce Walker, Fayetteville, AR (US); Brandon Beard, Tontitown, AR (US); Melissa Chanderban, Lowell, AR (US)

(73) Assignee: Jupeng Bio (HK) Limited, Hong Kong (CN)

(21) Appl. No.: 18/740,720

(22) Filed: Jun. 12, 2024

Related U.S. Application Data
(60) Provisional application No. 63/521,323, filed on Jun. 15, 2023.

(51) Int. Cl. C12P 5/02; C12M 1/107; C12N 1/06; C12N 1/20; C12P 7/06; C12R 1/145

(52) U.S. Cl. CPC C12P 5/023; C12M 21/04; C12N 1/066; C12N 1/20; C12P 7/065; C12R 2001/145

(57) ABSTRACT

System and process are provided for fixation of carbon dioxide through fermentation. More specifically, the disclosure includes fermenting carbon dioxide into methane through methanogenic archaea and producing single cell protein nutrient supplement. The disclosure further provides the integration of methanogenic fermentation with additional processes to achieve improved carbon efficiency.

Processes are provided for fixation of carbon dioxide. More specifically, the processes include fermenting carbon dioxide into methane and fermenting the methane with methylotrophic bacteria. Additional processes include processing cell mass from fermentations into single cell proteins to be used as nutrient supplements.

### BACKGROUND

Carbon monoxide and carbon dioxide emissions from industrial processes are two of the major drivers of climate change and global warming. Microbial fermentation can reduce such carbon emissions by utilizing microorganisms, through their metabolic pathways, to convert carbon monoxide (CO), hydrogen (H₂) and/or carbon dioxide (CO₂) into useful oxygenated hydrocarbon compounds, such as ethanol, butanol, acetate, butyrate, 2,3-butanediol, and other desired products.

Large scale microbial fermentation also produces large amount of microbial biomass. Traditionally, disposal of microbial biomass needs highly expensive waste treatment system, storage sites and landfills. Previously finding shows microbial biomass can be recovered into single cell protein (SCP) and other components for reuse as source of proteins, amino acids, and carbohydrates that are useful as a nutrient supplement for animals, plants, or human beings. For example, U.S. Pat. No. 10,856,560 describes a method of producing whole cell animal feed by culturing acetogens to produce microbial biomass.

Accordingly, there is a need for processes and systems which can effectively convert carbon dioxide into products for utilization in other processes. Further, there remains a need for a process and system for effectively converting microbial biomass into digestion friendly nutrient supplements, and compositions of any such nutrient supplements.

### SUMMARY

In one aspect, a process for converting CO₂ includes fermenting a gaseous substrate that includes CO₂ and H₂ with methanogenic archaea in a methanogen fermentation vessel to produce methane and a fermentation liquid broth containing methanogenic archaea and fermenting the methane with methylotrophic bacteria in a methylotrophic fermentation vessel to produce a fermentation liquid broth containing methylotrophic bacteria and a CO₂ containing vent gas.

In another aspect, a process for converting CO and CO₂ includes fermenting a gaseous substrate that includes CO₂ and H₂ with methanogenic archaea in a methanogen fermentation vessel to produce methane and a fermentation liquid broth containing methanogenic archaea. The methane is then fermented with methylotrophic bacteria in a methylotrophic fermentation vessel to produce a fermentation liquid broth containing methylotrophic bacteria and a first CO₂ containing vent gas. A gaseous substrate includes CO is fermented with CO converting acetogenic bacteria in a CO fermentation vessel to produce an alcohol, a second CO containing vent gas, and a fermentation liquid broth containing acetogenic bacteria.

### BRIEF DESCRIPTION OF FIGURES

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1A illustrates a process for converting CO₂ that includes methane production and fermentation with methylotrophic bacteria where single cell protein is processed together.

FIG. 1B illustrates a process for converting CO₂ that includes methane production and fermentation with methylotrophic bacteria where single cell protein is processed separately.

FIG. 2A shows a process for converting CO and CO₂ that includes methane production,, fermentation with CO converting acetogenic bacteria and fermentation with methylotrophic bacteria where single cell protein is processed together.

FIG. 2B shows a process for converting CO₂ that includes methane production, fermentation with CO converting acetogenic bacteria and fermentation with methylotrophic bacteria where single cell protein is processed separately.

### DETAILED DESCRIPTION

The following description is not to be taken in a limiting sense, but is made merely for the purpose of describing the general principles of exemplary embodiments. The scope of the disclosure should be determined with reference to the claims.

The term “about” modifying any amount refers to the variation in that amount encountered in real world conditions, e.g. in the lab, pilot plant, or production facility. For example, an amount of an ingredient or measurement employed in a mixture or quantity when modified by “about” includes the variation and degree of care typically employed in measuring in an experimental condition in production plant or lab. For example, the amount of a component of a product when modified by “about” includes the variation between batches in multiple experiments in the plant or lab and the variation inherent in the analytical method. Whether or not modified by “about” the amounts include equivalents to those amounts. Any quantity stated herein and modified by “about” can also be employed in the present disclosure as the amount not modified by “about”.

The use of the terms “a”, “an”, “the” and similar referents in the context of this disclosure are to be construed to cover both the singular and the plural, unless otherwise indicated or clearly contradicted by context.

Unless otherwise indicated, the terms “comprising”, “including”, “having”, “containing”, or “characterized by” are inclusive and does not exclude any additional, unrecited elements or method steps (i.e. meaning “including, but not limited to”). The use of any examples or exemplary language (e.g., “such as”, “for example”, “for instance”) provided herein is intended merely to illuminate the disclosure and does not impose a limitation on the scope of the disclosure unless otherwise claimed.

Fermentation is a metabolic process used by microorganism to generate energy for cell growth. Certain microorganism can ferment a Cl-containing gaseous substrate, such as syngas, carbon monoxide (CO) containing gaseous substrate, or carbon dioxide (CO₂) containing gaseous substrate, to sustain their growth and produce oxygenated hydrocarbon compounds. In such cases, the microorganism uses the one or more Cl components in the Cl-containing gaseous substrate as the primary carbon source for its growth. The terms “fermentation”, “fermentation process”, “microbial fermentation process” and the like are intended to encompass both the growth phase and the product biosynthesis phase of the process. During an anaerobic fermentation process, large amounts of microbial biomass are obtained, which may be purged out and processed into useful products, such as nutrient supplements. Specifically, the present disclosure includes a process of extracting nutrient supplements out of microbial biomass from an anaerobic fermentation process.

Fermentable gaseous substrate refers to CI-containing gaseous substrate comprises one or more of CO, CO₂, or CH₂O₂. Suitable gaseous substrate may include various synthesis gas (i.e. syngas) and industrial off-gas.

Syngas may be provided from any known source. In one aspect, syngas may be sourced from gasification of carbonaceous materials. Gasification involves partial combustion of biomass in a restricted supply of oxygen. The resultant gas may include CO, CO₂, and H₂. Some examples of suitable gasification methods and apparatus are provided in U.S. Ser. Nos. 61/516,667, 61/516,704 and 61/516,646, all of which were filed on Apr. 6, 2011, and in U.S. Ser. Nos. 13/427,144, 13/427,193 and 13/427,247, all of which were filed on Mar. 22, 2012, and all of which are incorporated herein by reference. In another aspect, syngas may be generated from electrolysis of water and carbon dioxide. In this aspect, oxygen is removed from the resultant gas and the resultant gas may be further blended with other gas sources to form a desired fermentable gaseous substrate.

Industrial off-gas may include the CI-containing waste gas from industrial processes that would otherwise be exhausted into the atmosphere. Examples of industrial off-gas include gases produced during microbial fermentation, ferrous metal products manufacturing, non-ferrous products manufacturing, petroleum refining processes, gasification of coal, electric power production, carbon black production, ammonia production, methanol production, coke manufacturing and gas reforming.

The Cl-containing gaseous substrate may include H₂. H₂ may also be separately supplemented into the Cl-containing gaseous substrate to form desired gas composition suitable for fermentation. Examples of H₂ sources include gases produced during ferrous metal products manufacturing, non-ferrous products manufacturing, petroleum refining processes, gasification of coal, gasification of biomass, electric power production, carbon black production, ammonia production, methanol production and coke manufacturing. Other sources of hydrogen may include for example, H₂O electrolysis and bio-generated H₂.

The fermentation of the fermentable gaseous substrate with the microorganism takes place in a fermentation vessel. Fermentation vessel includes a fermentation bioreactor consisting of one or more vessels and/or towers or piping arrangements, which includes a batch reactor, semi-batch reactor, continuous reactor, continuous stirred tank reactor (CSTR), bubble column reactor, external circulation loop reactor, internal circulation loop reactor, immobilized cell reactor (ICR), trickle bed reactor (TBR), moving bed biofilm reactor (MBBR), gas lift reactor, membrane reactor such as hollow fiber membrane bioreactor (HFMBR), static mixer, gas lift fermentor, or other vessel or other device suitable for gas-liquid contact.

A culture medium suitable for anaerobic microbial growth and fermenting fermentable gaseous substrate into one or more oxygenated hydrocarbon compounds can be added to the fermentation vessel to support the fermentation of the gaseous substrate by the acetogenic bacteria. Some examples of medium compositions are described in U.S. Ser. Nos. 16/530,502 and 16/530,481, filed Aug. 2, 2019, and in U.S. Pat. No. 7,285,402, filed Jul. 23, 2001, all of which are incorporated herein by reference. The medium may be sterilized to remove undesirable microorganisms and the fermentation vessel is inoculated with the desired microorganisms. Sterilization may not always be required. Suitable culture medium for methanogen fermentation is described in U.S. Pat. No. 11,401,499 which is incorporated herein by reference.

### Methanogenic Fermentation

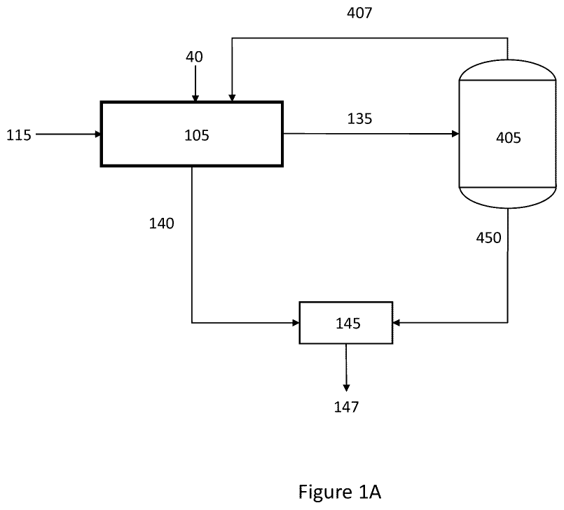

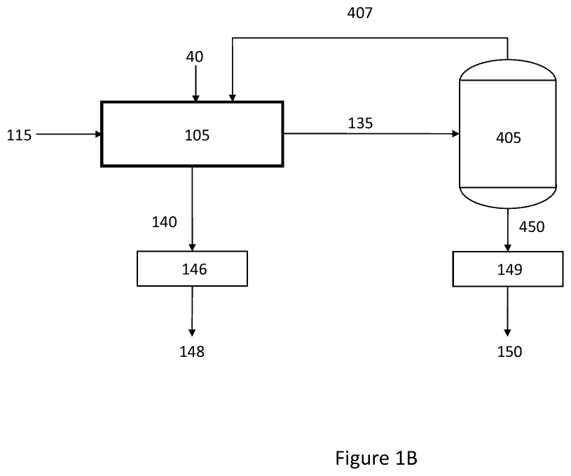

Methane Production: In one aspect illustrated in FIGS. 1A and 1B, a process includes a methanogen fermentation vessel 105 that can be integrated with industrial processes that produce CO₂. In this aspect, the methanogen fermentation vessel 105 contains a microbial culture capable of hydrogenotrophic methanogenesis (i.e. the conversion of CO: plus H₂ to methane). A separate hydrogen source 40 may be provided to the methanogen fermentation vessel 105. H₂ and CO₂ may each be added separately to the methanogen fermentation vessel 105 or blended together and then added to the methanogen fermentation vessel 105. The process includes maintaining a ratio of CO₂ to H₂ in the fermentation vessel 105 of about 1:5 to about 1:1, in another aspect, about 1:5 to about 1:2, in another aspect, about 1:5 to about 1:3, in another aspect, about 1:5 to about 1:4, in another aspect, about 1:4 to about 1:1, in another aspect, about 1:4 to about 1:2, in another aspect, about 1:4 to about 1:3, in another aspect, about 1:3 to about 1:1, in another aspect, about 1:3 to about 1:2, and in another aspect, about 1:2 to about 1:1. Total gas delivery rates in the range of about 0.2 to about 25 volume of gas, in another aspect, about 2 to about 16, in another aspect, about 1 to 22, and in another aspect, about 0.5 to 20 (STP, standard temperature and pressure) per volume of culture per minute are suitable. The methanogen fermentation vessel 105 may also produce a fermentation liquid broth containing methanogen archaea 140 that may be processed into single cell protein in a single cell protein processing unit 145 to produce a nutrient supplement 147.

Suitable microbial cultures are readily obtainable from public collections of organisms or can be isolated from a variety of environmental sources. Such environmental sources include anaerobic soils and sands, bogs, swamps, marshes, estuaries, dense algal mats, both terrestrial and marine mud and sediments, deep ocean and deep well sites, sewage and organic waste sites and treatment facilities, and animal intestinal tracts and feces. Many pure cultures of single species are suitable. Classified pure cultures are all members of the Archaeal domain [Woese et al. Proc Natl Acad Sci USA 87:4576-4579 (1990) “Towards a natural system of organisms: Proposal for the domains Archaea, Bacteria, and Eucharya.”, incorporated herein by reference] and fall within 4 different classes of the Euryarchaea kingdom. Examples of suitable organisms have been classified into 4 different genera within the Methanobacteria class (e.g. Methanobacterium alcaliphilum, Methanobacterium bryantii, Methanobacterium congolense, Methanobacterium defluvii, Methanobacterium espanolae, Methanobacterium formicicum, Methanobacterium ivanovii, Methanobacterium palustre, Methanobacterium thermaggregans, Methanobacterium uliginosum, Methanobrevibacter acididurans, Methanobrevibacter arboriphilicus, Methanobrevibacter gottschalkii, Methanobrevibacter olleyae. Methanobrevibacter ruminantium, Methanobrevibacter smithii, Methanobrevibacter woesei, Methanobrevibacter wolinii, Methanothermobacter marburgensis, Methanothermobacter thermautotrophicum (also known as Methanothermobacter thermoautotroiphicus), Methanothermobacter thermoflexus, Methanothermobacter thermophilus, Methanothermobacter wolfeii, Methanothermus sociabilis), 5 different genera within the Methanomicrobia class (e.g. Methanocorpusculum bavaricum, Methanocorpusculum parvum, Methanoculleus chikuoensis, Methanoculleus submarinus, Methanogenium frigidum, Methanogenium liminatans, Methanogenium marinum, Methanosarcina acetivorans, Methanosarcina barkeri, Methanosarcina mazei, Methanosarcina thermophila, Methanomicrobium mobile), 7 different genera within the Methanococci class (e.g. Methanocaldococcus jannaschii, Methanococcus aeolicus, Methanococcus maripaludis, Methanococcus vannielii, Methanococcus voltaei, Methanothermococcus thermolithotrophicus, Methanocaldococcus fervens, Methanocaldococcus indicus, Methanocaldococcus infernus, Methanocaldococcus vulcanius), and one genus within the Methanopyri class (e.g. Methanopyrus kandleri). Suitable cultures are available from public culture collections (e.g. the American Type Culture Collection, the Deutsche Sammlung von Mikroorganismen and Zellkulturen GmbH, and the Oregon Collection of Methanogens). Many suitable hydrogenotrophic methanogens, isolated in pure culture and available in public culture collections, have not yet been fully classified. Preferred pure culture organisms include Methanosarcinia barkeri. Methanococcus maripaludis, Methanothermobacter thermoautotrophicus, and Methanothermobacter marburgensis.

Suitable cultures of mixtures of two or more microbes are also readily isolated from the specified environmental sources [Bryant et al. Archiv Microbiol 59:20-31 (1967) “Methanobacillus omelianskii, a symbiotic association of two species of bacteria.”, incorporated herein by reference]. Suitable mixtures may be consortia in which cells of two or more species are physically associated or they may be syntrophic mixtures in which two or more species cooperate metabolically without physical association. Mixed cultures may have useful properties beyond those available from pure cultures of known hydrogenotrophic methanogens. These properties may include, for instance, resistance to contaminants in the gas feed stream, such as oxygen, ethanol or other trace components, or aggregated growth, which may increase the culture density and volumetric gas processing capacity of the culture.

Suitable cultures of mixed organisms may also be obtained by combining cultures isolated from two or more sources. One or more of the species in a suitable mixed culture should be an Archacal methanogen. Any non-Archaeal species may be bacterial or eukaryotic.

Suitable cultures may also be obtained by genetic modification of non-methanogenic organisms in which genes essential for supporting hydrogenotrophic methanogenesis are transferred from a methanogenic microbe or from a combination of microbes that may or may not be methanogenic on their own. Suitable genetic modification may also be obtained by enzymatic or chemical synthesis of the necessary genes.

The methanogen fermentation vessel 105 provides continuous methane production using a continuous hydrogenotrophic methanogenic culture operating under stable conditions. An example of such suitable conditions is provided in Schill, N., van Gulik, M., Voisard, D., & von Stockar, U. (1996) Biotechnol & Biocng 51:645-658. “Continuous cultures limited by a gaseous substrate: development of a simple, unstructured mathematical model and experimental verification with Methanobacterium thermoautotrophicum”, incorporated herein by reference. Culture media may be comprised of dilute mineral salts, and should be adapted to the particular culture in use.

Concentrations of various medium components for use in the methanogen fermentation process are as follows:

| | Concentration | feed Rate |
|---|---|---|
| Element | mg/L | μg/gram cells/min |
| NH₄⁺ | 82-3280 | 170-1,375 |
| Fe | 0.85-34 | 0.91-8.22 |
| Ni | 0.07-2.81 | 0.12-1.05 |
| Co | 0.037-1.49 | 0.13-0.66 |
| Zn | 0.45-23.8 | 0.08-0.56 |
| Mo | 0.003-0.397 | 0.012-0.13 |
| chelator | 2.5-100 | 2.65-17.37 |
| W | 0.8-32.1 | 0.94-8.06 |
| K | 98-3933 | 217-1,542 |
| Mg | 0.71-28.69 | 0.69-6.24 |
| Na | 875-35000 | 290-8,750 |
| S | 15-625 | 1.12-13.41 |
| P | 20-805 | 9.71-87.39 |

Medium in the methanogen fermentation vessel 105 should be replenished at a rate suitable to maintain a useful concentration of essential minerals and to eliminate any metabolic products that may inhibit methanogenesis. Dilution rates below 0.2 culture volume per hour are suitable, since they yield high volumetric concentrations of active methane generation capacity.

In one aspect, a redox potential is maintained below-400 mV or lower during methanogenesis. In another aspect, the redox potential is maintained below-300 mV or lower, in another aspect, below-200 mV, and in still another aspect, below-100 mV.

In another aspect, temperature of the culture is maintained near the optimum for growth of the organism used in the culture (e.g. about 35° C. to about 37° C. for mesophilic organisms such as Methanosarcinia barkeri and Methanococcus maripaludis or about 60°−65° C. for thermophiles such as Methanothermobacter thermoautotrophicus, and about 85° C. . . . 90° C. for organisms such as Methanocaldococcus jannaschii, Methanocaldococcus fervens, Methanocaldococcus indicus, Methanocaldococcus infernus, and Methanocaldococcus vulcanius.). However, it is envisioned that temperatures above or below the temperatures for optimal growth may be used.

In another aspect, a reducing agent may be introduced into the fermentation process along with CO₂ and H₂. This reducing agent can suitably be hydrogen sulfide or sodium sulfide. Hydrogen itself can be used as a reductant to maintain the redox potential of the culture in the range (<−100 mV) necessary for optimum performance of hydrogenotrophic methanogenesis. Generally, hydrogen gas is provided in concentrations effective in allowing for at least a portion of the carbon dioxide in the bioreactor to be converted into methane. In another aspect, the redox potential of the culture can be maintained at <−100 mV via an electrochemical cell immersed in the medium.

In another aspect, the process includes various methods and/or features that reduce the presence of oxygen in the CO₂ stream that is fed into the bioreactor. When obligate anaerobic methanogenic archaea are used to catalyze methane formation, the presence of oxygen may be detrimental to the performance of the process and contaminates the product gas. Therefore, the reduction of the presence of oxygen in the CO₂ stream is helpful for improving the process. In one aspect, the oxygen level is reduced prior to entry of the gas into the fermentation vessel by passing the mixed H₂/CO₂ stream over a palladium catalyst, which converts any trace oxygen to water. In this aspect, H₂ is provided in an amount above the amount needed in the culture by a 2:1 ratio relative to the contaminating oxygen. In another aspect, the oxygen is removed by pre-treatment of the gas stream in a bioreactor. In this aspect, the reductant may be provided either by provision of a source of organic material (e.g. glucose, starch, cellulose, fermentation residue from an ethanol plant, whey residue, etc.) that can serve as substrate for an oxidative fermentation. The microbial biological catalyst is chosen to oxidatively ferment the chosen organic source, yielding CO₂ from the contaminant oxygen. In this embodiment, additional He would be provided to enable conversion in the anaerobic fermentor of this additional CO₂ to methane.

The current process provides a specific CO; uptake of about 0.5 to about 3 mmol CO₂/minute/gram of cells, in another aspect, about 1 to about 2 mmol CO₂/minute/gram of cells, in another aspect, about 0.5 to about 1 mmol CO₂/minute/gram of cells, in another aspect, about 1 to about 3 mmol CO₂/minute/gram of cells, and in another aspect, about 0.5 to about 2 mmol CO₂/minute/gram of cells. In this aspect, the current process is effective for providing a CO₂ conversion rate of 65% or more, in another aspect, 70% or more, in another aspect, 75% or more, in another aspect, 80% or more, in another aspect, 85% or more, in another aspect, 90% or more, in another aspect, 85% to 95%, and in another aspect, 90% to 99%.

The process further provides a cell density of up to 100 g/L, in one aspect, 10 to 80 g/L, in one aspect, 15 to 60 g/L, in one aspect, 20 to 50 g/L, in one aspect 10 to 30 g/L, and in another aspect, 15 to 45 g/L.

The process further provides a specific H₂ uptake of about 3 to about 12 mmol H₂/minute/gram of cells, in another aspect, about 3 to about 10 mmol H₂/minute/gram of cells, in another aspect, about 3 to about 8 mmol H₂/minute/gram of cells, in another aspect, about 3 to about 6 mmol H₂/minute/gram of cells, in another aspect, about 4 to about 12 mmol H₂/minute/gram of cells, in another aspect, about 4 to about 10 mmol H₂/minute/gram of cells, in another aspect, about 4 to about 8 mmol H₂/minute/gram of cells, in another aspect, about 5 to about 12 mmol H₂/minute/gram of cells, in another aspect, about 5 to about 10) mmol H₂/minute/gram of cells, and in another aspect, about 5 to about 8 mmol H₂/minute/gram of cells.

The process further provides a cell retention time of about 5 to about 50 hours, in another aspect, about 5 to about 40 hours, in another aspect, about 5 to about 30 hours, in another aspect, about 5 to about 25 hours, in another aspect, about 5 to about 20 hours, in another aspect, about 5 to about 10 hours, in another aspect, about 5 to about 8 hours, and in another aspect, about 8 to about 15 hours.

The process further provides a methane productivity of about 0.4 to about 3 mmol methane/minute/gram of cells, in another aspect, about 0.4 to about 2 mmol methane/minute/gram of cells, in another aspect, in another aspect, about 0.4 to about 1 mmol methane/minute/gram of cells, in another aspect, about 1.0 to about 3 mmol methane/minute/gram of cells, in another aspect, about 1.0 to about 2.5 mmol methane/minute/gram of cells, in another aspect, about 1.0 to about 2.5 mmol methane/minute/gram of cells, and in another aspect, about 1.5 to about 2.5 mmol methane/minute/gram of cells. In this aspect, the process is effective for providing a methane effluent gas with more than 60% methane concentration, in another aspect, more than 65% methane concentration, in another aspect, more than 70% methane concentration, in another aspect, more than 75%, in another aspect, more than 80%, and in still another aspect, more than 85%.

### Methanogenic Fermentation and Methylotrophic Fermentation

A process for converting CO₂ as illustrated in FIGS. 1A and 1B includes methane production and use of methylotrophic fermentation 405 to convert the methane 135 into a fermentation liquid broth containing methylotrophic bacteria 450 that may be processed into single cell protein nutrient supplement. Fermentation liquid broth containing methanogenic archaea cells 140 may also be processed into single cell protein. As shown in FIG. 1A methylotrophic bacteria and methanogenic archaea may be processed in a single cell protein processing unit 145 to produce a nutrient supplement 147. In an alternative aspect shown in FIG. 1B, methanogenic archaea may be processed in a first single cell protein processing unit 146 to produce a first nutrient supplement 148, and methylotrophic bacteria may be processed in a second single cell protein processing unit 149 to produce a second nutrient supplement 150.

Methane Production: In one aspect illustrated in FIGS. 1A and 1B, a process includes a methanogen fermentation vessel 105 as described herein with the description of methanogenic fermentation. A supplemented hydrogen stream 40 may also be provided to the methane fermentation vessel 105. CO₂ produced in methylotrophic vessel 405 may be recycled to the methanogen fermentation vessel through line 407.

Methylotrophic fermentation: As illustrated in FIGS. 1A and 1B, methane 135 may be provided to a methylotrophic fermentation vessel 405. The methylotrophic fermentation vessel 405 includes methylotrophs. Methylotrophs are a diverse group of microorganisms that can use reduced one-carbon compounds, such as methanol or methane, as their carbon source for their growth. Methylotrophs cells 450 can then be processed into single cell proteins. Examples of methylotrophs include Methylomonas methanica, Methylosinus trichosporium, and Methylococcus capsulatus, Methylobacterium exiorquens, Paracoccus denitrificans, Methylomicrobium alcaliphilum, Methylacidiphilum fumariolicum, Methylomicrobium buryatense, and Methanoperedens nitroreducens. Genetically modified organisms capable of using methane may also be utilized. Examples of suitable growth conditions for methylotrophs are provided in U.S. Pat. No. 10,934,566 and US Publication No. US20210340574 which are both incorporated herein by reference.

In methylotrophic fermentation, methane and oxygen are converted into cell mass and CO₂. The CO₂ containing vent gas may be then provided to the methanogen fermentation vessel 105 as a feed gas. Oxygen in the CO₂ containing vent gas is removed before entering the methanogen fermentation vessel.

### Co Fermentation, Methanogenic Fermentation, and Methylotrophic Fermentation

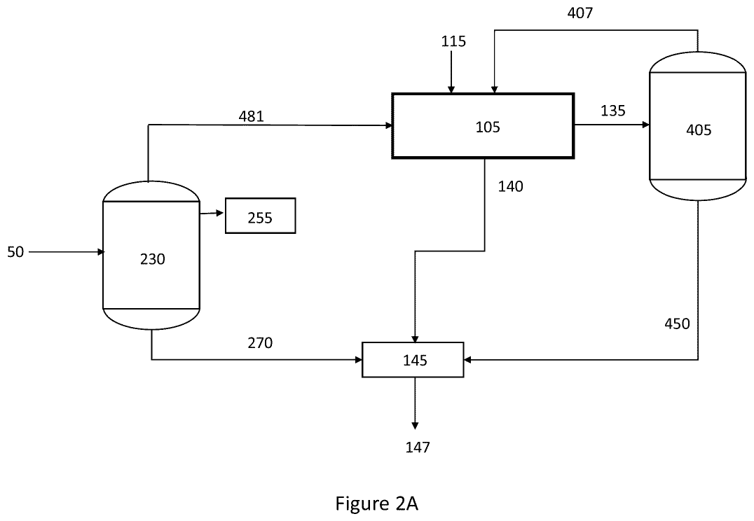

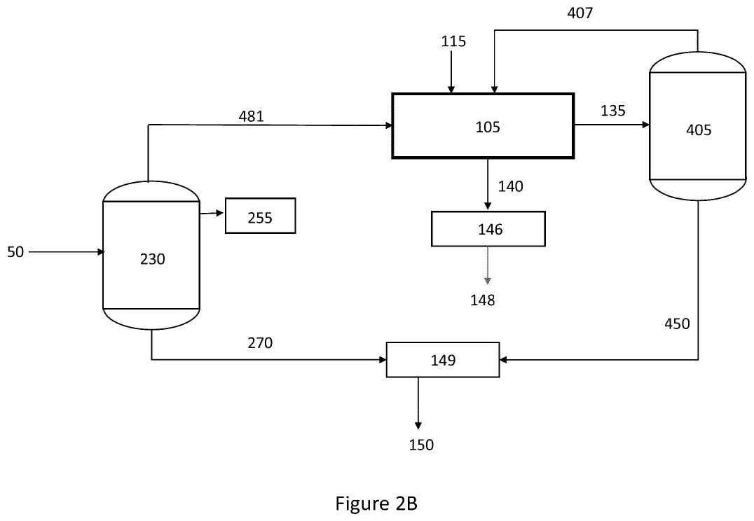

Methane Production: In one aspect illustrated in FIGS. 2A and 2B, a process includes a methanogen fermentation vessel 105 as described herein with the description of FIGS. 1A and 1B.

CO Fermentation: Certain acetogenic bacteria can ferment CO-containing gaseous substrate 50 in a CO fermentation vessel 230 into useful oxygenated hydrocarbon compounds 255, such as ethanol and butanol, and produce a fermentation liquid broth containing the acetogenic bacteria 270.

In this aspect, suitable gaseous substrate 50 contains at least about 5 mole % CO, in one aspect, at least about 10 mole %, in one aspect, at least about 20 mole %, in one aspect, at least about 30 mole %, in one aspect, about 10 to about 100 mole %, in another aspect, about 20 to about 100 mole % CO, in another aspect, about 30 to about 90 mole % CO, in another aspect, about 40 to about 80 mole % CO, and in another aspect, about 50 to about 70 mole % CO. In this aspect, the CO-containing gaseous substrate 50 may have about 40 mole % or less CO₂., in one aspect, the CO-containing gaseous substrate 50 may have about 30 mole % or less CO₂., in one aspect, the CO-containing gaseous substrate 50 may have about 20 mole % or less CO₂., in another aspect, the CO-containing gaseous substrate 50 may have about 10 mole % or less CO₂., in another aspect, the CO-containing gaseous substrate 50 may have about 1 mole % or less CO₂, in still another aspect, the CO-containing gaseous substrate 50 may comprise no or substantially no CO₂.

Depending on the composition of the CO-containing gaseous substrate 50, the CO-containing gaseous substrate 50 may be directly provided to the fermentation vessel 230 or may be further modified or blended to include an appropriate H₂ to CO molar ratio. In one aspect, the CO-containing gaseous substrate provided to the fermentation vessel has an H₂ to CO molar ratio of about 0.1 or more, in another aspect, about 0.2 or more, in another aspect, about 0.25 or more, and in another aspect, about 0.5 or more. In one aspect, a second portion of H₂ from a methane cracker may be supplied to the CO fermentation vessel 230.

Concentrations of various medium components for use in the CO bioconversion fermentation process are as follows:

| | Concentration | Feed Rate |
|---|---|---|
| Element | mg/L | μg/gram of cells/min |
| NH₄⁺ | 164-6560 | 41-1640 |
| Fe | 1.7-68 | 0.425-17 |
| Ni | 0.07-2.81 | 0.017-0.702 |
| Co | 0.037-1.49 | 0.009-0.373 |
| Se | 0.027-1.1 | 0.006-0.274 |
| Zn | 0.116-4.64 | 0.198-5.95 |
| W | 0.8-32.1 | 0.26-8.03 |
| K | 39-1573 | 9.83-393.25 |
| Mg | 1.4-57.3 | 0.35-14.32 |
| S | 15-625 | 3.9-156.2 |
| P | 15-601 | 3.76-150.43 |
| d-biotin | 0.016-0.64 | 0.004-0.16 |
| thiamine HCl | 0.04-1.6 | 0.01-0.4 |
| calcium-D-pantothenate | 0.02-0.81 | 0.005-0.202 |

Examples of useful acetogenic bacteria for CO bioconversion fermentation process include Blautia producta, Butyribacterium methylotrophicum, Caldanaerobacter subterraneous, Caldanaerobacter subterraneous pacificus, Carboxydothermus hydrogenoformans, Clostridium aceticum, Clostridium acetobutylicum, Clostridium acetobutylicum P262, Clostridium autoethanogenum (DSM 19630 of DSMZ Germany), Clostridium autoethanogenum (DSM 10061 of DSMZ Germany), Clostridium autoethanogenum (DSM 23693 of DSMZ Germany), Clostridium autoethanogenum (DSM 24138 of DSMZ Germany), Clostridium carboxidivorans, Clostridium coskatii (ATCC PTA-10522), Clostridium drakei, Clostridium ljungdahlii PETC (ATCC 49587), Clostridium ljungdahlii ERI2 (ATCC 55380), Clostridium ljungdahlii C-01 (ATCC 55988), Clostridium ljungdahlii 0-52 (ATCC 55889), Clostridium magnum, Clostridium pasteurianum (DSM 525 of DSMZ Germany), Clostridium ragsdalei PII (ATCC BAA-622), Clostridium scatologenes, Clostridium thermoaceticum, Clostridium ultunense, Desulfotomaculum kuznetsovii, Eubacterium limosum, Geobacter sulfurreducens, Methanosarcina acetivorans, Methanosarcina barkeri, Oxobacter pfennigii, Peptostreptococcus productus Clostridium Stick-landii, and mixtures thereof.

Anaerobic bacteria are bacteria that do not require oxygen for growth. An anaerobic bacteria may react negatively or even die if oxygen is present above certain threshold. Acetogenic bacteria are microorganisms that are capable of producing acetate under anaerobic respiration or fermentation by utilizing the Wood-Ljungdahl pathway as their main mechanism for energy conservation. Other useful oxygenated hydrocarbon compounds, such as formic acid, propionic acid, butyric acid, heptanoic acid, decanoic acid, ethanol, butanol, 2-butanol, and 2,3-butanediol, may also be produced by the acetogenic bacteria. Examples of the acetogenic bacteria suitable for converting CI-containing gaseous substrate to useful oxygenated hydrocarbon compounds include those of the genus Clostridium, such as strains of Clostridium ljungdahlii, including those described in WO 2000/68407, EP 117309, U.S. Pat. Nos. 5,173,429, 5,593,886 and 6,368,819, WO 1998/00558 and WO 2002/08438, strains of Clostridium autoethanogenum (DSM 10061 and DSM 19630 of DSMZ, Germany) including those described in WO 2007/117157 and WO 2009/151342 and Clostridium ragsdalei (P11, ATCC BAA-622) and Alkalibaculum bacchi (CP11, ATCC BAA-1772) including those described respectively in U.S. Pat. No. 7,704,723 and “Biofuels and Bioproducts from Biomass-Generated Synthesis Gas”, Hasan Atiyeh, presented in Oklahoma EPSCOR Annual State Conference, Apr. 29, 2010 and Clostridium carboxidivorans (ATCC PTA-7827) described in U.S. Patent Application No. 2007/0276447. Other suitable microorganisms includes those of the genus Moorella, including Moorella sp. HUC22-1, and those of the genus Carboxydothermus. Each of these references is incorporated herein by reference.

The CO fermentation may desirably be carried out under appropriate reaction conditions for the desired fermentation mode. For example, in one aspect, the CO fermentation can be set at a mode that focuses on CO-to-oxygenated hydrocarbon compounds (e.g. ethanol) production. In this mode, about 4% to 6% of the carbon from the CO fed to the CO fermentation is converted to biomass. In another aspect, the CO fermentation may be set at a mode that focuses on CO-to-microbial biomass production. In this mode, about 6% to 7.5% of the carbon from the CO fed to the CO fermentation is converted to biomass. Reaction conditions that should be considered include pressure, temperature, gas flow rate, liquid flow rate, medium pH, medium redox potential, agitation rate (if using a stirred tank reactor), inoculum level, appropriate gas substrate concentrations to ensure that CO in the liquid phase does not become limiting nor inhibitory, and appropriate product concentrations to avoid product inhibition. The CO fermentation process further provides a CO conversion rate of 80% or more, in one aspect, 85% or more, in one aspect, 90% or more, in another aspect, 80% to 99%, in another aspect, 85% to 98%, and in still another aspect 90% to 97%.

The CO converting acetogenic bacteria converts CO and produces one or more alcohols and a CO₂ containing vent gas. The CO₂ containing vent gas is then sent to the methanogenic fermentation vessel. The CO₂ containing vent gas contains 5% or less CO, in one aspect, 3% or less, in one aspect, 2% or less, and in another aspect, 1% or less. CO may be removed from the CO₂ containing vent gas before it enters the methanogenic fermentation vessel to avoid CO inhibition.

A process for converting CO and CO₂ as illustrated in FIGS. 2A and 2B includes fermenting a gaseous substrate 50 that includes CO in a CO fermentation vessel 230. CO converting acetogenic bacteria in the CO fermentation vessel 230 may convert CO into one or more alcohols 255. Vent gas 481 from the CO fermentation vessel 230 contains CO₂. The vent gas 481 is provided to a methanogenic fermentation vessel 105. A gaseous substrate 115 containing additional H₂ and/or CO₂ may also be provided to the methanogen fermentation vessel 105. Methanogenic archaea in the methanogen fermentation vessel convert CO₂ into methane 135. Methane 135 may be provided to a methylotrophic fermentation vessel 405. The methylotrophic fermentation vessel 405 includes methylotrophs bacteria, which consumes methane for microbial growth. CO fermentation broth containing CO converting acctogenic bacteria cells 270, methylotrophic fermentation broth containing methylotrophic bacteria cells 450, and methanogen fermentation broth containing methanogenic archaea cells 140 can be further processed into single cell protein nutrient supplements.

Methylotrophic fermentation: As illustrated in FIGS. 2A and 2B and further described in FIGS. 1A and 1B, methane 135 may be provided to a methylotrophic fermentation vessel 405. The methylotrophic fermentation vessel 405 includes methylotrophs.

### Integrated Fermentation Systems

As would be understood by a person of ordinary skill in the art, any one or all of the processes described in FIGS. 1A, 1B, 2A, and 2B may be combined into an overall system.

### Microbial Biomass

The fermentation liquid broth (FIGS. 1A and 1B: 140 and 450, FIGS. 2A and 2B: 140, 270 and 450) from any of the fermentation vessels may be further purged out of the fermentation vessel and then processed into protein containing nutrient supplement in one or more single cell protein processing unit. Fermentation liquid broth may be processed separately or together as shown in the Figures. The fermentation liquid broth is separated into a cell-free permeate and a cell-containing suspension by one or more cell separators. Cell membranes and/or cell walls of the cells in the cell-containing suspension are ruptured to generate a homogenate. The homogenate is then fractionated into a protein-containing supernatant and a protein-containing cell debris portion by using a fractionator.

Suitable cell separators include, but not limited to, filtration devices, hollow fiber filtration devices, spiral wound filtration devices, ultrafiltration devices, ceramic filter devices, cross-flow filtration devices, size exclusion column filtration devices, spiral wound membranes, centrifugation devices, and combination thereof. Processes for production of single cell proteins from biomass are described in U.S. Ser. No. 16/416,133, filed May 17, 2019.

The cell-containing suspension contains microbial cells at a cell concentration higher than the fermentation liquid broth. In one aspect, the cell concentration of the cell-containing suspension is about 20 g/L or more, in another aspect, about 30 g/L or more, in another aspect, about 40 g/L or more, in another aspect, about 50 g/L or more, in another aspect, about 60 g/L or more, in another aspect, about 20 to about 300 g/L, in another aspect, about 30 to about 250 g/L, in another aspect, about 40 to about 200 g/L, in another aspect, about 50 to about 150 g/L, in still another aspect, about 100 to about 150 g/L.

Cells of the cell-containing suspension may be ruptured using one or more rupturing devices selected from the group consisting of a microfluidics device, a sonication device, an ultrasonic device, a mechanical disruption device, a French press, a freezer, a heater, a heat exchanger, a distillation column, a pasteurization device, an UV sterilization device, a gamma ray sterilization device, a reactor, a homogenizer, and combinations thereof. In one aspect, a pH of the cell-containing suspension is adjusted to a pH of about 6 to about 12 before rupturing cell membranes of the cell-containing suspension, in another aspect, a pH of 7 to 12, in another aspect, a pH of 8 to 12, in another aspect, a pH of 7.5 to 11, in another aspect, a pH of 8.5 to 11, and in still another aspect, a pH of 7 to 10.

In another aspect, cell-containing suspension is hydrolyzed by a hydrolase enzyme. In this aspect, the cell-containing suspension and the hydrolase enzyme are incubated at a temperature of about 50) to about 70° C. for about 3 to about 72 hours to form a hydrolyzed lysate, in one aspect, 3 to 48 hours, in one aspect, 4 to 24 hours, in one aspect, 6 to 24 hours, in another aspect, 6 to 12 hours, and in still another aspect, 4 to 12 hours. pH of the cell-containing suspension is adjusted to a pH of about 6 to about 12 before hydrolysis of the cell-containing suspension, in another aspect, a pH of 7 to 12, in another aspect, a pH of 8 to 12, in another aspect, a pH of 7.5 to 11, in another aspect, a pH of 8.5 to 11, and in still another aspect, a pH of 7 to 10. The hydrolase enzyme is selected from the group consisting of subtilases, alcalase, serine protease, serine endopeptidase and mixtures thereof. The hydrolyzed lysate is fractionated into the protein-containing supernatant and the protein-containing cell debris portion using centrifugation, ultrafiltration, and combination thereof. The protein-containing supernatant has a nucleic acid content of less than about 5%, in one aspect, less than 4%, in one aspect, less than 3%, and in another aspect, less than 2%.

The protein-containing supernatant and the protein-containing cell debris portion may be directly used as or further processed to a protein containing nutrient supplement. A dehydration unit may be used to dry the protein-containing supernatant and produce a soluble protein containing nutrient supplement, such as protein powder. Suitable dehydration unit includes spray drying unit, drum dryer unit, freeze drying unit, lyophilizing unit, and combinations thereof. Other components, such as moisture and ash can be further removed to purify the protein containing supplement. The protein containing supplement may be directly used as animal feed or be blended with other ingredients for making into one or more types of nutrient supplements. In one aspect, the protein containing supplement contains about 60 to about 99 weight percent protein, in another aspect, about 70 to about 95 weight percent protein, in another aspect, about 75 to about 95 weight percent protein, in another aspect, about 80 to 95 weight percent protein, and in another aspect, about 85 to 95 weight percent protein.

The processes for producing protein containing nutrient supplement from methylotrophic bacteria and acetogenic bacteria may differ from producing protein containing nutrient supplement from methanogenic archaea due to a lack of cell wall in the methanogenic archaea. As shown in FIGS. 1B and 2B, nutrient supplement 148 from methanogenic archaea may be produced in a single cell protein processing unit 146 while the nutrient supplement 150 produced from methylotrophic bacteria and/or acetogenic bacteria may be produced in another single cell protein processing unit 149. In such scenario, the operating cost and processing time in the single cell protein processing unit 146 may be significantly lower than the single cell protein processing unit 149.

### EXAMPLES

The following examples further illustrate the disclosure and should not be construed to limit its scope.

### Example 1: Methanogen Fermentation of CO₂

A gas containing CO₂ and H₂ is continuously introduced into a stirred tank bioreactor containing Methanothermobacter thermautotrophicus, along with a conventional liquid medium containing trace metals and salts.

New Brunswick Bioflow 320 reactor containing Fermentation Medium is started with actively growing Methanothermobacter thermautotrophicus. The rate of agitation of the reactor is set to 1200 rpm at the start of the experiment. This agitation rate is maintained throughout the experiment. Feed gas flow to the reactor is increased based on the H₂ and CO₂ uptake of the culture. Temperature in the bioreactor is maintained around 60° C. throughout the experiment. Samples of gas feed into the bioreactor and off-gas from the bioreactor and fermentation broth in the bioreactor are taken at intervals, for example feed gas, off-gas and fermentation broth are sampled about daily, once two hours and once four hours respectively. Above samples are analyzed for consumption or production of various gas components, and the optical density (cell density) of the culture. The unaroused volume of the reactor is maintained in 2000 to 2200 ml throughout the experiment. Also, the gas flow to the reactor is measured real time by the mass flow controller regulating gas to the reactor. The feed gas composition of this experiment is 76% H₂, 20% CO₂ and 4% N₂.

In this experiment, a cell recycle system (CRS) is attached to the reactor before the start of the experiment. During the experiment, the rate of flow of nutrients (growth medium) to the reactor is 2.0-5.0 ml/min. Rate of cell purge is 3.0 to 5.0 ml/min and permeate is drawn out through the CRS at 0 to 2.0 ml/min.

Results can be summarized as follows:

Specific CO₂ uptake: 0.8-1.1 mmol CO₂/min/gram of cells

Specific H₂ uptake: 3.3-3.8 mmol H₂/min/gram of cells

Average Cell Retention Time: 11.2 hours

Average Cell Density: 3 g/L

CO₂ conversion rate: 90% to 99%

Specific methane productivity is 0.77 mmol/min/gram of cells

The effluent gas composition of this experiment is 13.8% H₂, 7.6% CO₂, 62.5% CH₄ and 16.1% N₂

While the disclosure herein disclosed has been described by means of specific embodiments, examples, and applications thereof, other and further variations could be devised without departing from the basic scope of the disclosure set forth in the claims that follow.

What is claimed is:

1. A process for converting CO₂, the process comprising:
fermenting a gaseous substrate that includes CO, and H₂ with methanogenic archaea in a methanogen fermentation vessel to produce methane and a fermentation liquid broth containing methanogenic archaea; and
fermenting the methane with methylotrophic bacteria in a methylotrophic fermentation vessel to produce a fermentation liquid broth containing methylotrophic bacteria and a CO₂ containing vent gas.

2. The process of claim 1 wherein at least a portion of the CO₂ containing vent gas is sent to the methanogen fermentation vessel.

3. The process of claim 2 wherein at least a portion of O₂ in the CO₂ containing vent gas is removed before the at least a portion of the CO₂ containing vent gas is sent to the methanogen fermentation vessel.

4. The process of claim 1 wherein the gaseous substrate that includes CO₂ and H₂ has a CO₂ to H₂ ratio of about 1:3 to 1:4.

5. The process of claim 1 wherein the methanogenic archaea is selected from the group consisting of Methanobacterium alcaliphilum, Methanobacterium bryantii, Methanobacterium congolense, Methanobacterium defluvii, Methanobacterium espanolae, Methanobacterium formicicum, Methanobacterium ivanovii, Methanobacterium palustre, Methanobacterium thermaggregans, Methanobacterium uliginosum, Methanobrevibacter acididurans, Methanobrevibacter arboriphilicus, Methanobrevibacter gottschalkii, Methanobrevibacter olleyae, Methanobrevibacter ruminantium, Methanobrevibacter smithii, Methanobrevibacter woesei, Methanobrevibacter wolinii, Methanothermobacter marburgensis, Methanothermobacter thermautotrophicum, Methanothermobacter thermoflexus, Methanothermobacter thermophilus, Methanothermobacter wolfeii, Methanothermus sociabilis, Methanocorpusculum bavaricum, Methanocorpusculum parvum, Methanoculleus chikuoensis, Methanoculleus submarinus, Methanogenium frigidum, Methanogenium liminatans, Methanogenium marinum, Methanosarcina acetivorans, Methanosarcina barkeri, Methanosarcina mazei, Methanosarcina thermophila, Methanomicrobium mobile, Methanocaldococcus jannaschii, Methanococcus acolicus, Methanococcus maripaludis, Methanococcus vannielii, Methanococcus voltaei, Methanothermococcus thermolithotrophicus, Methanopyrus kandleri, Methanothermobacter thermoautotroiphicus, Methanocaldococcus fervens, Methanocaldococcus indicus, Methanocaldococcus infernus, and Methanocaldococcus vulcanius.

6. The process of claim 1 wherein the methanogenic archaea is Methanothermobacter thermoautotroiphicus.

7. The process of claim 1 wherein the methanogen fermentation vessel provides a specific CO₂ uptake of about 0.5 to about 3 mmol CO₂/minute/gram of cells.

8. The process of claim 1 wherein the methanogen fermentation vessel provides a specific H₂ uptake of about 1.5 to about 12 mmol H₂/minute/gram of cells.

9. The process of claim 1 wherein the methanogen fermentation vessel has a cell retention time of about 5 to about 50 hours.

10. The process of claim 1 wherein the methanogen fermentation vessel provides a methane productivity of about 0.5 to about 2.5 mmol methane/minute/gram of cells.

11. The process of claim 1 wherein the methanogen fermentation vessel provides a CO₂ conversion rate of 65% or more.

12. The process of claim 1, wherein the methylotrophic bacteria is selected from the group consisting of Methylomicrobium alcaliphilum, Methylacidiphilum fumariolicum, Methylomicrobium buryatense, Methanoperedens nitroreducens, and combinations thereof.

13. The process of claim 1, further comprising:
separating the fermentation liquid broth containing methylotrophic bacteria into a first cell-free permeate and a first cell-containing suspension;
rupturing cell membranes of cells in the first cell-containing suspension to generate a first homogenate;
fractionating the first homogenate into a first protein-containing supernatant and a first protein-containing cell debris portion; and
recovering a first protein containing nutrient supplement.

14. The process of claim 13 wherein the first cell-containing suspension has a dry cell weight concentration of about 20 g/liter to about 200 g/liter.

15. The process of claim 13 wherein rupturing cell membranes of the first cell-containing suspension is conducted using one or more rupturing devices selected from the group consisting of a microfluidics device, a sonication device, an ultrasonic device, a mechanical disruption device, a French press, a freezer, a heater, a heat exchanger, a distillation column, a pasteurization device, an UV sterilization device, a gamma ray sterilization device, a reactor, a homogenizer, and combinations thereof.

16. The process of claim 13 wherein a pH of the first cell-containing suspension is adjusted to a pH of about 6 to about 12 before rupturing cell membranes of the first cell-containing suspension.

17. The process of claim 13 wherein the first homogenate is a hydrolyzed lysate formed by contacting the first cell-containing suspension with a hydrolase enzyme.

18. The process of claim 17 wherein the first cell-containing suspension and the hydrolase enzyme are incubated at a temperature of about 50 to about 70° C. for about 3 to about 72 hours to form the hydrolyzed lysate.

19. The process of claim 17 wherein the hydrolase enzyme is selected from the group consisting of subtilases, alcalase, serine protease, serine endopeptidase and mixtures thereof.

20. The process of claim 13 wherein the first homogenate is fractionated into the first protein-containing supernatant and the first protein-containing cell debris portion using centrifugation, ultrafiltration, and combination thereof.

21. The process of claim 13 wherein the first protein-containing supernatant has a nucleic acid content of less than about 5%.

22. The process of claim 13 wherein the first protein-containing supernatant is dehydrated to provide a first soluble protein containing nutrient supplement with about 60 to about 99 dry weight percent protein.

23. The process of claim 1, further comprising:
separating the fermentation liquid broth containing methanogenic archaca into a second cell-free permeate and a second cell-containing suspension;
rupturing cell membranes of cells in the second cell-containing suspension to generate a second homogenate;
fractionating the second homogenate into a second protein-containing supernatant and a second protein-containing cell debris portion; and
recovering a second protein containing nutrient supplement.

24. The process of claim 23 wherein the second cell-containing suspension has a dry cell weight concentration of about 20 g/liter to about 200 g/liter.

25. The process of claim 23 wherein rupturing cell membranes of the second cell-containing suspension is conducted using one or more rupturing devices selected from the group consisting of a microfluidics device, a sonication device, an ultrasonic device, a mechanical disruption device, a French press, a freezer, a heater, a heat exchanger, a distillation column, a pasteurization device, an UV sterilization device, a gamma ray sterilization device, a reactor, a homogenizer, and combinations thereof.

26. The process of claim 23 wherein a pH of the second cell-containing suspension is adjusted to a pH of about 6 to about 12 before rupturing cell membranes of the second cell-containing suspension.

27. The process of claim 23 wherein the second homogenate is a hydrolyzed lysate formed by contacting the second cell-containing suspension with a hydrolase enzyme.

28. The process of claim 27 wherein the second cell-containing suspension and the hydrolase enzyme are incubated at a temperature of about 50 to about 70° C. for about 4 to about 24 hours to form the hydrolyzed lysate.

29. The process of claim 27 wherein the hydrolase enzyme is selected from the group consisting of subtilases, alcalase, serine protease, serine endopeptidase and mixtures thereof.

30. The process of claim 23 wherein the second homogenate is fractionated into the second protein-containing supernatant and the second protein-containing cell debris portion using centrifugation, ultrafiltration, and combination thereof.

31. The process of claim 23 wherein the second protein-containing supernatant has a nucleic acid content of less than about 5%.

32. The process of claim 23 wherein the second protein-containing supernatant is dehydrated to provide a second soluble protein containing nutrient supplement with about 60 to about 99 dry weight percent protein.

33. The process of claim 1, further comprising:
mixing the fermentation liquid broth containing methylotrophic bacteria with the fermentation liquid broth containing methanogenic archaea to generate a mixed cell containing fermentation liquid broth;
separating the mixed cell containing fermentation liquid broth into a third cell-free permeate and a third cell-containing suspension;
rupturing cell membranes of cells in the third cell-containing suspension to generate a third homogenate;
fractionating the third homogenate into a third protein-containing supernatant and a third protein-containing cell debris portion; and
recovering a third protein containing nutrient supplement.

34. The process of claim 33 wherein the third cell-containing suspension has a dry cell weight concentration of about 20 g/liter to about 200 g/liter.

35. The process of claim 33 wherein rupturing cell membranes of the third cell-containing suspension is conducted using one or more rupturing devices selected from the group consisting of a microfluidics device, a sonication device, an ultrasonic device, a mechanical disruption device, a French press, a freezer, a heater, a heat exchanger, a distillation column, a pasteurization device, an UV sterilization device, a gamma ray sterilization device, a reactor, a homogenizer, and combinations thereof.

36. The process of claim 33 wherein a pH of the third cell-containing suspension is adjusted to a pH of about 6 to about 12 before rupturing cell membranes of the third cell-containing suspension.

37. The process of claim 33 wherein the third homogenate is a hydrolyzed lysate formed by contacting the third cell-containing suspension with a hydrolase enzyme.

38. The process of claim 37 wherein the third cell-containing suspension and the hydrolase enzyme are incubated at a temperature of about 50 to about 70° C. for about 3 to about 72 hours to form the hydrolyzed lysate.

39. The process of claim 37 wherein the hydrolase enzyme is selected from the group consisting of subtilases, alcalase, serine protease, serine endopeptidase and mixtures thereof.

40. The process of claim 33 wherein the third homogenate is fractionated into the third protein-containing supernatant and the third protein-containing cell debris portion using centrifugation, ultrafiltration, and combination thereof.

41. The process of claim 33 wherein the third protein-containing supernatant has a nucleic acid content of less than about 5%.

42. The process of claim 33 wherein the third protein-containing supernatant is dehydrated to provide a third soluble protein containing nutrient supplement with about 60 to about 99 dry weight percent protein.

43. A process for converting CO and CO₂, the process comprising:
fermenting a gaseous substrate that includes CO₂ and H₂ with methanogenic archaea in a methanogen fermentation vessel to produce methane and a fermentation liquid broth containing methanogenic archaea;
fermenting the methane with methylotrophic bacteria in a methylotrophic fermentation vessel to produce a fermentation liquid broth containing methylotrophic bacteria and a first CO₂ containing vent gas; and
fermenting a gaseous substrate that includes CO with CO converting acetogenic bacteria in a CO fermentation vessel to produce an alcohol, a second CO₂ containing vent gas, and a fermentation liquid broth containing acetogenic bacteria.

44. The process of claim 43 wherein at least a portion of the first CO₂ containing vent gas is sent to the methanogen fermentation vessel.

45. The process of claim 44 wherein at least a portion of O₂ in the first CO₂ containing vent gas is removed before the at least a portion of the first CO₂ containing vent gas is sent to the methanogen fermentation vessel.

46. The process of claim 43 wherein at least a portion of the second CO₂ containing vent gas is sent to the methanogen fermentation vessel.

47. The process of claim 46 wherein at least a portion of CO in the second CO₂ containing vent gas is removed before the at least a portion of the second CO: containing vent gas is sent to the methanogen fermentation vessel.

48. The process of claim 43 wherein the gaseous substrate that includes CO₂ and H₂ includes at least a portion of the first CO₂ containing vent gas, at least a portion of the second CO₂ containing vent gas, or the combination of at least a portion of the first CO₂ containing vent gas and at least a portion of the second CO₂ containing vent gas.

49. The process of claim 43 wherein the gaseous substrate that includes CO; and H₂ has a CO₂ to H₂ ratio of about 1:3 to 1:4.

50. The process of claim 43, wherein the methanogenic archaea is selected from the group consisting of Methanobacterium alcaliphilum, Methanobacterium bryantii, Methanobacterium congolense, Methanobacterium defluvii, Methanobacterium espanolae, Methanobacterium formicicum, Methanobacterium ivanovii, Methanobacterium palustre, Methanobacterium thermaggregans, Methanobacterium uliginosum, Methanobrevibacter acididurans, Methanobrevibacter arboriphilicus, Methanobrevibacter gottschalkii, Methanobrevibacter olleyae, Methanobrevibacter ruminantium, Methanobrevibacter smithii, Methanobrevibacter woesei, Methanobrevibacter wolinii, Methanothermobacter marburgensis, Methanothermobacter thermautotrophicum, Methanothermobacter thermoflexus, Methanothermobacter thermophilus, Methanothermobacter wolfeii, Methanothermus sociabilis, Methanocorpusculum bavaricum, Methanocorpusculum parvum, Methanoculleus chikuoensis, Methanoculleus submarinus, Methanogenium frigidum, Methanogenium liminatans, Methanogenium marinum, Methanosarcina acetivorans, Methanosarcina barkeri, Methanosarcina mazei, Methanosarcina thermophila, Methanomicrobium mobile, Methanocaldococcus jannaschii, Methanococcus aeolicus, Methanococcus maripaludis, Methanococcus vannielii, Methanococcus voltaei, Methanothermococcus thermolithotrophicus, Methanopyrus kandleri, Methanothermobacter thermoautotroiphicus, Methanocaldococcus fervens, Methanocaldococcus indicus, Methanocaldococcus infernus, and Methanocaldococcus vulcanius.

51. The process of claim 43 wherein the methanogenic archaea is Methanothermobacter thermoautotroiphicus.

52. The process of claim 43 wherein the methanogen fermentation provides a specific CO₂ uptake of about 1 to about 3 mmol CO₂/minute/gram of cells.

53. The process of claim 43 wherein the methanogen fermentation provides a specific H₂ uptake of about 3 to about 12 mmol H₂/minute/gram of cells.

54. The process of claim 43 wherein the methanogen fermentation has a cell retention time of about 5 to about 50 hours.

55. The process of claim 43 wherein the methanogen fermentation provides a methane productivity of about 1.2 to about 2.5 mmol methane/minute/gram of cells.

56. The process of claim 43 wherein the methanogen fermentation vessel provides a CO₂ conversion rate of 65% or more.

57. The process of claim 43 wherein the CO converting acetogenic bacteria is selected from the group consisting of Clostridium aceticum, Clostridium acetobutylicum, Clostridium acetobutylicum P262, Clostridium autoethanogenum (DSM 19630 of DSMZ Germany), Clostridium autoethanogenum (DSM 10061 of DSMZ Germany), Clostridium autoethanogenum (DSM 23693 of DSMZ Germany), Clostridium autoethanogenum (DSM 24138 of DSMZ Germany), Clostridium carboxidivorans, Clostridium coskatii (ATCC PTA-10522), Clostridium drakei, Clostridium ljungdahlii PETC (ATCC 49587), Clostridium ljungdahlii ERI2 (ATCC 55380), Clostridium ljungdahlii C-01 (ATCC 55988), Clostridium ljungdahlii 0-52 (ATCC 55889), Clostridium magnum, Clostridium pasteurianum (DSM 525 of DSMZ Germany), Clostridium ragsdalei PII (ATCC BAA-622), Clostridium scatologenes, Clostridium thermoaceticum, Clostridium ultunense, Clostridium Stick-landii, and mixtures thereof.

58. The process of claim 43 wherein the alcohol is ethanol.

59. The process of claim 43 wherein the CO fermentation vessel provides a CO conversion rate of 80% or more.

60. The process of claim 43 wherein the methylotrophs are selected from the group consisting of Methylomicrobium alcaliphilum, Methylacidiphilum fumariolicum, Methylomicrobium buryatense, Methanoperedens nitroreducens, and combinations thereof.

61. The process of claim 43, further comprising:
separating the fermentation liquid broth containing methanogenic archaea into a first cell-free permeate and a first cell-containing suspension;
rupturing cell membranes of cells in the first cell-containing suspension to generate a first homogenate;
fractionating the first homogenate into a first protein-containing supernatant and a first protein-containing cell debris portion; and
recovering a first protein containing nutrient supplement.

62. The process of claim 61 wherein the second cell-containing suspension has a dry cell weight concentration of about 20 g/liter to about 200 g/liter.

63. The process of claim 61 wherein rupturing cell membranes of the first cell-containing suspension is conducted using one or more rupturing devices selected from the group consisting of a microfluidics device, a sonication device, an ultrasonic device, a mechanical disruption device, a French press, a freezer, a heater, a heat exchanger, a distillation column, a pasteurization device, an UV sterilization device, a gamma ray sterilization device, a reactor, a homogenizer, and combinations thereof.

64. The process of claim 61 wherein a pH of the first cell-containing suspension is adjusted to a pH of about 6 to about 12 before rupturing cell membranes of the first cell-containing suspension.

65. The process of claim 61 wherein the first homogenate is a hydrolyzed lysate formed by contacting the first cell-containing suspension with a hydrolase enzyme.

66. The process of claim 65 wherein the first cell-containing suspension and the hydrolase enzyme are incubated at a temperature of about 50 to about 70° C. for about 4 to about 24 hours to form the hydrolyzed lysate.

67. The process of claim 65 wherein the hydrolase enzyme is selected from the group consisting of subtilases, alcalase, serine protease, serine endopeptidase and mixtures thereof.

68. The process of claim 61 wherein the first homogenate is fractionated into the first protein-containing supernatant and the first protein-containing cell debris portion using centrifugation, ultrafiltration, and combination thereof.

69. The process of claim 61 wherein the first protein-containing supernatant has a nucleic acid content of less than about 5%.

70. The process of claim 61 wherein the first protein-containing supernatant is dehydrated to provide a first soluble protein containing nutrient supplement with about 60 to about 99 dry weight percent protein.

71. The process of claim 43, further comprising:
mixing the fermentation liquid broth containing methylotrophic bacteria with the fermentation liquid broth containing acetogenic bacteria to generate a first mixed cell containing fermentation liquid broth;
separating the first mixed cell containing fermentation liquid broth into a second cell-free permeate and a second cell-containing suspension;
rupturing cell membranes of cells in the second cell-containing suspension to generate a second homogenate;
fractionating the second homogenate into a second protein-containing supernatant and a second protein-containing cell debris portion; and
recovering a second protein containing nutrient supplement.

72. The process of claim 71 wherein the second cell-containing suspension has a dry cell weight concentration of about 20 g/liter to about 200 g/liter.

73. The process of claim 71 wherein rupturing cell membranes of the second cell-containing suspension is conducted using one or more rupturing devices selected from the group consisting of a microfluidics device, a sonication device, an ultrasonic device, a mechanical disruption device, a French press, a freezer, a heater, a heat exchanger, a distillation column, a pasteurization device, an UV sterilization device, a gamma ray sterilization device, a reactor, a homogenizer, and combinations thereof.

74. The process of claim 71 wherein a pH of the second cell-containing suspension is adjusted to a pH of about 6 to about 12 before rupturing cell membranes of the second cell-containing suspension.

75. The process of claim 71 wherein the second homogenate is a hydrolyzed lysate formed by contacting the second cell-containing suspension with a hydrolase enzyme.

76. The process of claim 75 wherein the second cell-containing suspension and the hydrolase enzyme are incubated at a temperature of about 50 to about 70° C. for about 3 to about 72 hours to form the hydrolyzed lysate.

77. The process of claim 75 wherein the hydrolase enzyme is selected from the group consisting of subtilases, alcalase, serine protease, serine endopeptidase and mixtures thereof.

78. The process of claim 71 wherein the second homogenate is fractionated into the second protein-containing supernatant and the second protein-containing cell debris portion using centrifugation, ultrafiltration, and combination thereof.

79. The process of claim 71 wherein the second protein-containing supernatant has a nucleic acid content of less than about 5%.

80. The process of claim 71 wherein the second protein-containing supernatant is dehydrated to provide a second soluble protein containing nutrient supplement with about 60 to about 99 dry weight percent protein.

81. The process of claim 43, further comprising:
mixing the fermentation liquid broth containing methylotrophic bacteria and the fermentation liquid broth containing methanogenic archaea with the fermentation liquid broth containing acetogenic bacteria to generate a second mixed cell containing fermentation liquid broth;
separating the second mixed cell containing fermentation liquid broth into a third cell-free permeate and a third cell-containing suspension;
rupturing cell membranes of cells in the third cell-containing suspension to generate a third homogenate;
fractionating the third homogenate into a third protein-containing supernatant and a third protein-containing cell debris portion; and
recovering a third protein containing nutrient supplement.

82. The process of claim 81 wherein the third cell-containing suspension has a dry cell weight concentration of about 20 g/liter to about 200 g/liter.

83. The process of claim 81 wherein rupturing cell membranes of the third cell-containing suspension is conducted using one or more rupturing devices selected from the group consisting of a microfluidics device, a sonication device, an ultrasonic device, a mechanical disruption device, a French press, a freezer, a heater, a heat exchanger, a distillation column, a pasteurization device, an UV sterilization device, a gamma ray sterilization device, a reactor, a homogenizer, and combinations thereof.

84. The process of claim 81 wherein a pH of the third cell-containing suspension is adjusted to a pH of about 6 to about 12 before rupturing cell membranes of the third cell-containing suspension.

85. The process of claim 81 wherein the third homogenate is a hydrolyzed lysate formed by contacting the third cell-containing suspension with a hydrolase enzyme.

86. The process of claim 85 wherein the third cell-containing suspension and the hydrolase enzyme are incubated at a temperature of about 50 to about 70° C. for about 3 to about 72 hours to form the hydrolyzed lysate.

87. The process of claim 85 wherein the hydrolase enzyme is selected from the group consisting of subtilases, alcalase, serine protease, serine endopeptidase and mixtures thereof.

88. The process of claim 81 wherein the third homogenate is fractionated into the third protein-containing supernatant and the third protein-containing cell debris portion using centrifugation, ultrafiltration, and combination thereof.

89. The process of claim 81 wherein the third protein-containing supernatant has a nucleic acid content of less than about 5%.

90. The process of claim 81 wherein the third protein-containing supernatant is dehydrated to provide a third soluble protein containing nutrient supplement with about 60 to about 99 dry weight percent protein.
